(19) United States
(12) Patent Application Publication
(10) Pub. No.: US 2020/0288945 A1
(43) Pub. Date: Sep. 17, 2020

(54) INSERTION APPARATUS, CONNECTION STRUCTURE OF EXTERIOR MEMBER, AND ENDOSCOPE

(71) Applicant: OLYMPUS CORPORATION, Tokyo (JP)

(72) Inventors: Mitsuru KANEKO, Tokyo (JP); Shohei UEHARA, Tokyo (JP); Reiji KOYAMA, Kawagoe-shi (JP)

(73) Assignee: OLYMPUS CORPORATION, Tokyo (JP)

(21) Appl. No.: 16/887,199

(22) Filed: May 29, 2020

Related U.S. Application Data
(63) Continuation of application No. PCT/JP2018/034477, filed on Sep. 18, 2018.

(30) Foreign Application Priority Data
Nov. 30, 2017 (JP) 2017-230644

(51) Int. Cl. A61B 1/00; A61B 1/005

(52) U.S. Cl. CPC A61B 1/00071; A61B 1/00128; A61B 1/0051; A61B 1/00137

(57) ABSTRACT

An insertion apparatus includes a first exterior member, a second exterior member, and a sealing member disposed at a connection part and being configured to deform elastically, and a convex portion disposed in a spaced-apart manner from the sealing member in a longitudinal axis direction or in a circumferential direction, and being configured to position the first exterior member with respect to the second exterior member in the longitudinal axis direction and in the circumferential direction.

### CROSS REFERENCE TO RELATED APPLICATION

This application is a continuation application of PCT/JP2018/034477 filed on Sep. 18, 2018 and claims benefit of Japanese Application No. 2017-230644 filed in Japan on Nov. 30, 2017, the entire contents of which are incorporated herein by this reference.

### BACKGROUND OF THE INVENTION

### 1. Field of the Invention

The present invention relates to an insertion apparatus which includes a first exterior member, and a second exterior member disposed at a position where the second exterior member is adjacent to the first exterior member in a longitudinal axis direction of the first exterior member, a connection structure of an exterior member, and an endoscope.

### 2. Description of the Related Art

Recently, an insertion apparatus such as an endoscope has been widely used in the medical field and the industrial field.

With respect to an endoscope, the endoscope having the following structure is well known, that is, the endoscope including: an insertion portion which is inserted into a subject; an operation portion which is connected to a proximal end of the insertion portion (hereinafter, simply referred to as a proximal end) in a direction along a longitudinal axis of the insertion portion (hereinafter, simply referred to as a longitudinal axis direction); a universal cord which extends from the operation portion; and a connector which is mounted on an extending end of the universal cord and is detachably connected to an external apparatus.

In the endoscope having such a configuration, with respect to an exterior of the endoscope, for example, an exterior of the operation portion of the endoscope, there has been well known a configuration where the exterior is formed by connecting a plurality of exterior members along the above-mentioned longitudinal axis.

For example, there has been well known a configuration where an exterior of an operation portion is formed by connecting along the longitudinal axis a first exterior member formed in a barrel shape and a second exterior member disposed at a position adjacently to the first exterior member in a longitudinal axis direction and formed in a barrel shape.

More specifically, there has also been well known a structure where a first exterior member and a second exterior member are connected to each other in a state where a small diameter part having a barrel shape and a narrow diameter which is formed on a distal end side of the second exterior member in the longitudinal axis direction (hereinafter, simply referred to as a distal end side) is inserted into the -first exterior member, and an end surface of a part of the second exterior member having a diameter which changes is made to abut against a proximal end surface of the first exterior member along the longitudinal axis so that the end surface is positioned along the longitudinal axis.

There has been also well known a structure where a watertight state of a connection part is maintained by bringing a water tightness maintaining member such as an O-shaped ring mounted on an outer peripheral surface of a small diameter part into contact with an inner peripheral surface of a first exterior member.

Further, in Japanese Patent Application Laid-Open Publication No. 2006-130238, a configuration is disclosed where an elastically deformable sealing member is filled in a gap at a connection part between exterior members, and the gap between the exterior members is sealed by collapsing the sealing member between both exterior members along a longitudinal axis.

In the above-mentioned configuration where the first exterior member and the second exterior member are connected to each other, it is necessary to provide a positioning member to at least one of the first exterior member and the second exterior member so as to prevent the occurrence of a phenomenon that the first exterior member is rotated with respect to the second exterior member in a circumferential direction, or a phenomenon that the first exterior member is displaced with respect to the second exterior member along the longitudinal axis for controlling a compression amount of the sealing member.

This is because when the position of the first exterior member with respect to the second exterior member is displaced in the longitudinal axis direction and in the circumferential direction, accuracy in collapsing the sealing member (a control of a compression amount) between both exterior members becomes unstable and hence, an external appearance of a connection part does not become smooth and exhibits poor appearance.

In view of such circumstances, for example, there has been also well known a configuration where positioning between both exterior members is performed by making a convex portion formed on an outer peripheral surface of a small diameter part and a recessed portion formed on an inner peripheral surface of the first exterior member engage with each other by fitting.

### SUMMARY OF THE INVENTION

An insertion apparatus according to an aspect of the present invention includes: a first exterior member; a second exterior member disposed at a position where the second exterior member is adjacent to the first exterior member in a longitudinal axis direction of the first exterior member; a sealing member disposed at a connection part between the first exterior member and the second exterior member in the longitudinal axis direction, the sealing member being configured to deform elastically; and a positioning member disposed in a spaced-apart manner from the sealing member in the longitudinal axis direction or in a circumferential direction around a longitudinal axis of the first exterior member, the positioning member configured to position the first exterior member with respect to the second exterior member in the longitudinal axis direction and in the circumferential direction.

A connection structure of an exterior member according to an aspect of the present invention includes: a first exterior member; a second exterior member disposed at a position where the second exterior member is adjacent to the first exterior member in a longitudinal axis direction of the first exterior member; a sealing member disposed at a connection part between the first exterior member and the second exterior member in the longitudinal axis direction, the sealing member being configured to deform elastically; and a positioning member disposed in a spaced-apart manner from the sealing member in the longitudinal axis direction or in a circumferential direction around a longitudinal axis of the first exterior member, the positioning member configured to position the first exterior member with respect to the second exterior member in the longitudinal axis direction and in the circumferential direction.

An endoscope according to an aspect of the present invention includes: an operation portion which includes a first exterior member, a second exterior member disposed at a position where the second exterior member is adjacent to the first exterior member in a longitudinal axis direction of the first exterior member, and a sealing member disposed at a connection part between the first exterior member and the second exterior member in the longitudinal axis direction, the sealing member being configured to deform elastically; and an insertion portion connected to the operation portion on a first exterior member side, wherein the first exterior member and the second exterior member include a positioning member disposed in a spaced-apart manner from the sealing member in the longitudinal axis direction or in a circumferential direction around a longitudinal axis of the first exterior member, the positioning member configured to position the first exterior member with respect to the second exterior member in the longitudinal axis direction and in the circumferential direction.

### BRIEF DESCRIPTION OF THE DRAWINGS

FIG. 1 is a view showing an external appearance of an endoscope according to an embodiment;

FIG. 2 is a plan view showing an exterior of an operation portion in FIG. 1 in an enlarged manner;

FIG. 3 is a cross-sectional view of the exterior of the operation portion taken along a line III-III in FIG. 2;

FIG. 4 is a cross-sectional view of the exterior of the operation portion taken along a line IV-IV in FIG. 3;

FIG. 5 is a cross-sectional view of the exterior of the operation portion taken along a line V-V in FIG. 2;

FIG. 6 is an enlarged cross-sectional view of a part in FIG. 5 surrounded by a line VI;

FIG. 7 is an enlarged cross-sectional view of a part in FIG. 3 surrounded by a line VII;

FIG. 8 is an enlarged cross-sectional view of a part in FIG. 7 surrounded by a line VIII;

FIG. 9 is a cross-sectional view of the exterior of the operation portion taken along a line IX-IX in FIG. 7;

FIG. 10 is a partial perspective view schematically showing a recessed portion formed on an inner peripheral surface of a distal end of a small diameter part of a second exterior member in FIG. 7;

FIG. 11 is a partial perspective view schematically showing an end portion side of a convex portion in FIG. 7 which engages with the recessed portion in FIG. 10; and

FIG. 12 is a partial perspective view schematically showing a state where the convex portion in FIG. 11 engages with the recessed portion in FIG. 10.

### DETAILED DESCRIPTION OF THE PREFERRED EMBODIMENT

Hereinafter, an embodiment of the present invention is described with reference to drawings. Drawings are schematic views. Accordingly, it should be kept in mind that a relationship between a thickness and a width of each member, a ratio between thicknesses of respective members and the like differ from the corresponding relationships of members of an actual insertion apparatus. Needless to say, portions of the insertion apparatus are described with different size relationships or different ratios between the drawings.

In the embodiment described hereinafter, the description is made by taking an endoscope as an example of an insertion apparatus.

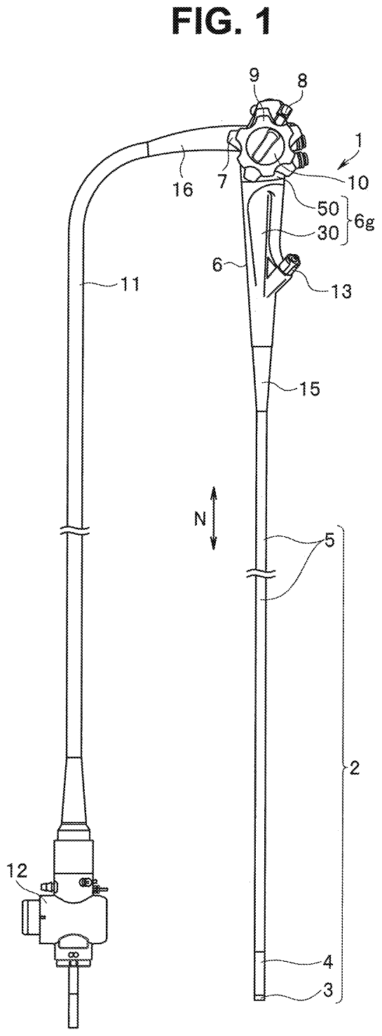

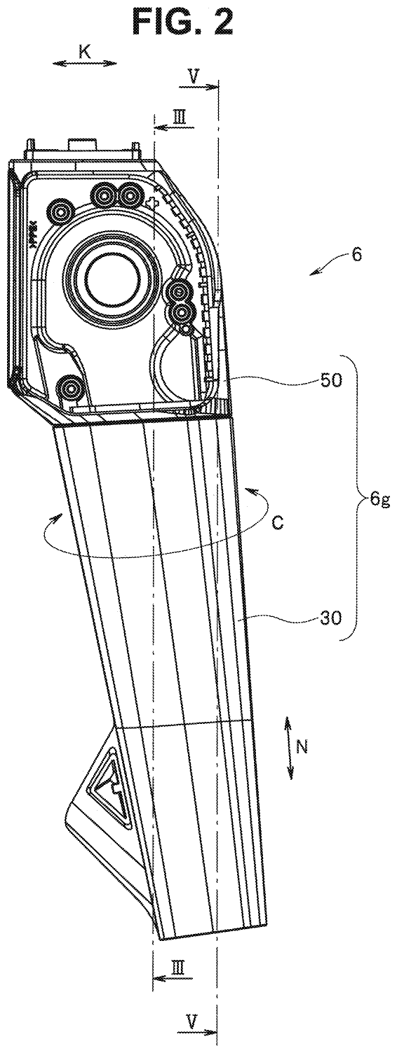

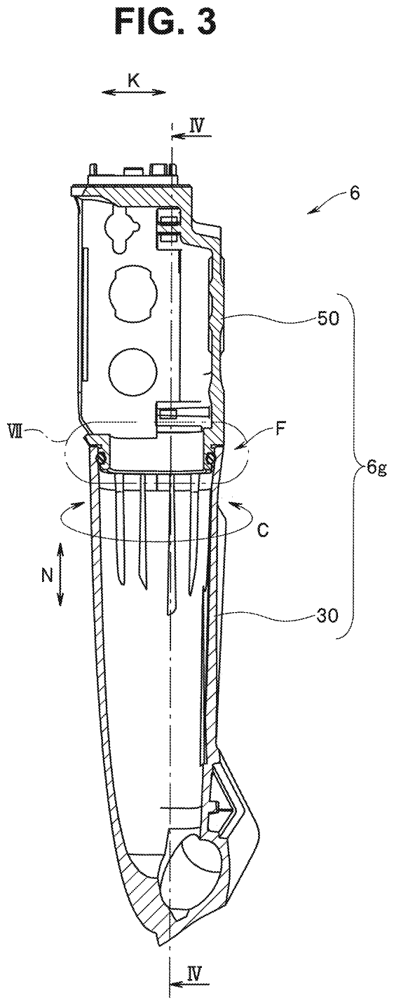

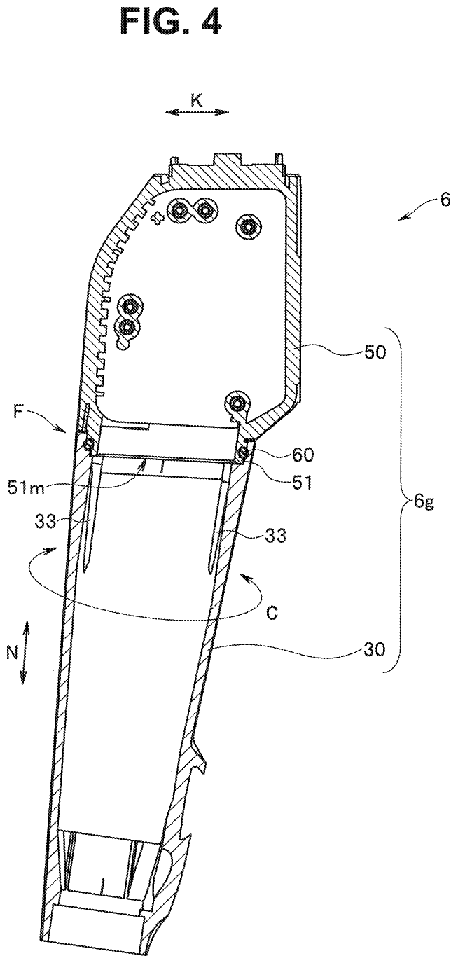

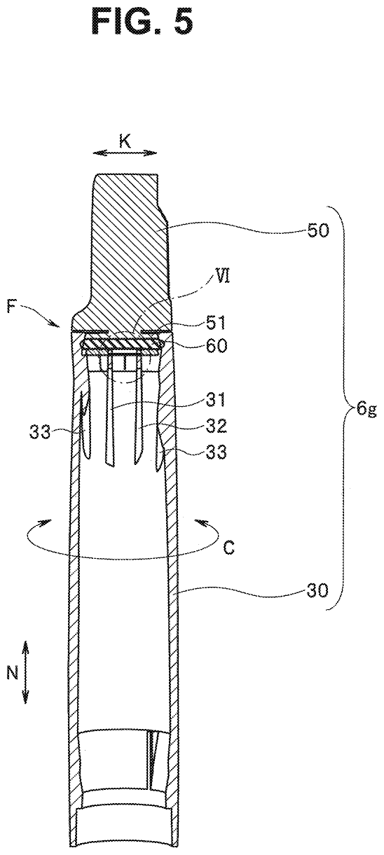

FIG. 1 is a view showing an external appearance of an endoscope according to this embodiment. FIG. 2 is a plan view showing an exterior of an operation portion in FIG. 1 in an enlarged manner. FIG. 3 is a cross-sectional view of the exterior of the operation portion taken along a line III-III in FIG. 2. FIG. 4 is a cross-sectional view of the exterior of the operation portion taken along a line IV-IV in FIG. 3. FIG. 5 is a cross-sectional view of the exterior of the operation portion taken along a line V-V in FIG. 2.

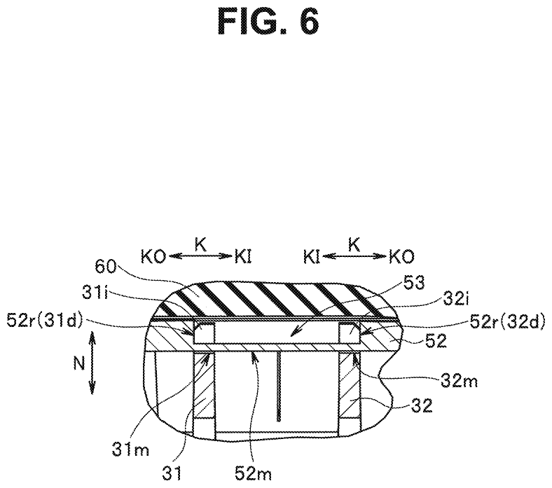

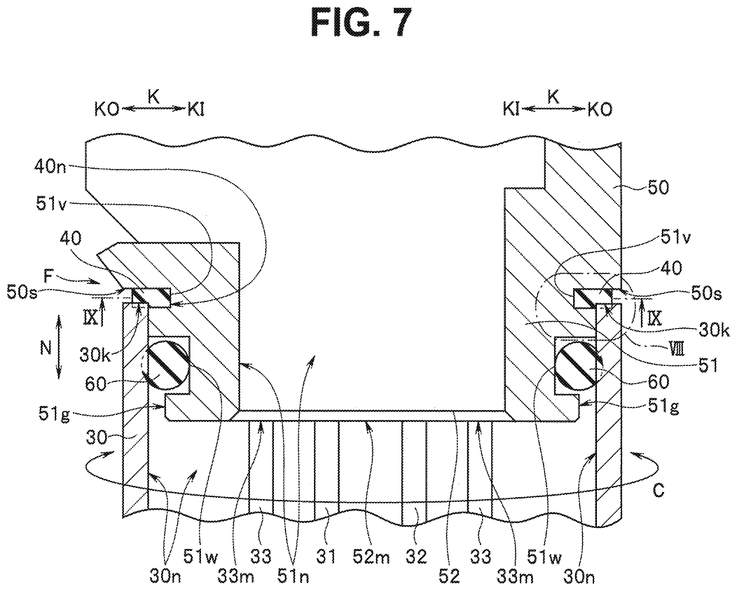

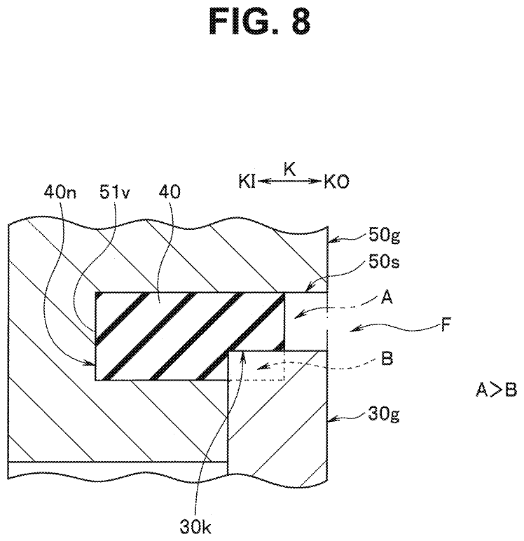

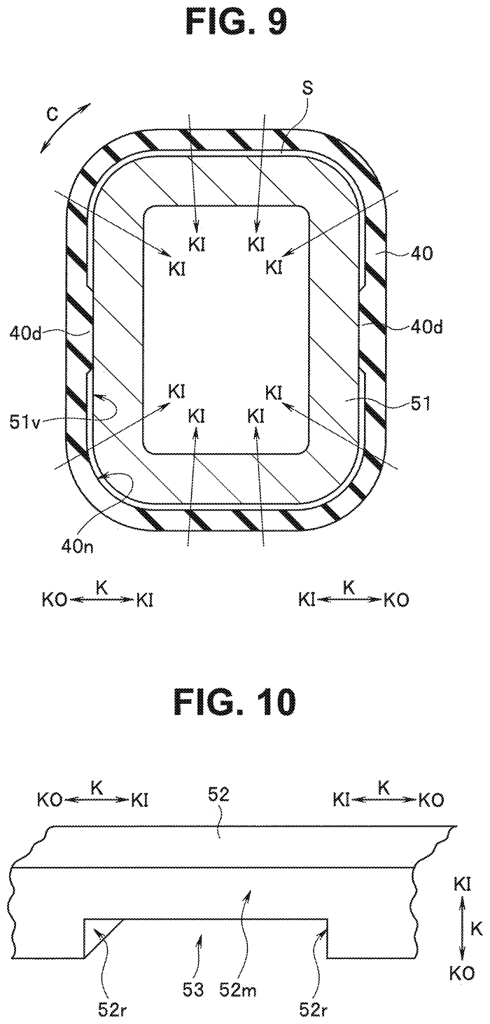

FIG. 6 is an enlarged cross-sectional view of a part in FIG. 5 surrounded by a line VI. FIG. 7 is an enlarged cross-sectional view of a part in FIG. 3 surrounded by a line VII. FIG. 8 is an enlarged cross-sectional view of a part in FIG. 7 surrounded by a line VIII. FIG. 9 is a cross-sectional view of the exterior of the operation portion taken along a line IX-IX in FIG. 7.

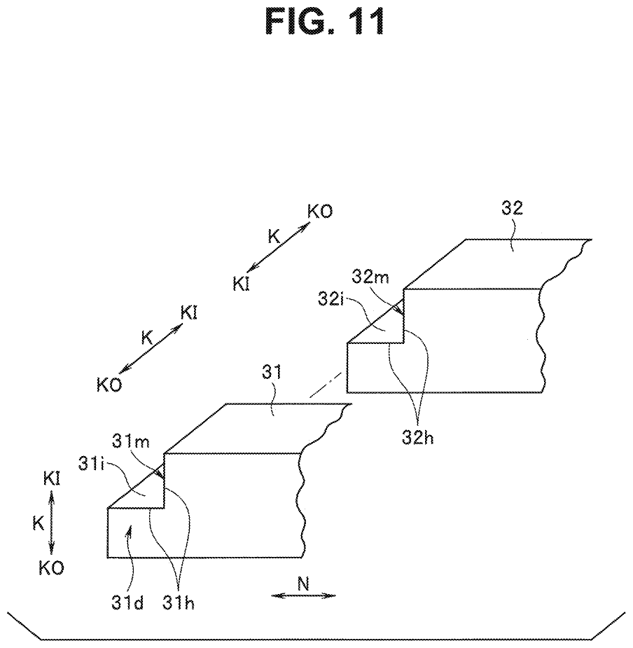

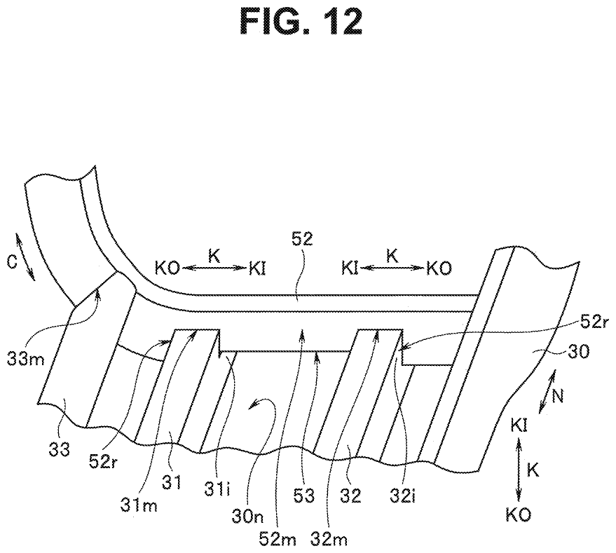

FIG. 10 is a partial perspective view schematically showing a recessed portion formed on an inner peripheral surface of a distal end of a small diameter part of a second exterior member in FIG. 7. FIG. 11 is a partial perspective view schematically showing an end portion side of a convex portion in FIG. 7 which engages with the recessed portion in FIG. 10. FIG. 12 is a partial perspective view schematically showing a state where the convex portion in FIG. 11 engages with the recessed portion in FIG. 10.

As shown in FIG. 1, a main part of the endoscope 1 is formed of: an insertion portion 2 which has flexibility, is inserted into a subject, and extends in a longitudinal axis direction N which is a direction of a longitudinal axis; an operation portion 6 which is connected to a proximal end of the insertion portion 2 by way of a bend preventing member 15 in the longitudinal axis direction N; a universal cord 11 which extends from the operation portion 6 by way of a bend preventing member 16; and a connector 12 which is mounted on an extending end of the universal cord 11.

The endoscope 1 is electrically connected to external apparatuses such as a controller and an illumination apparatus by way of the connector 12.

A treatment instrument insertion opening 13 is formed on the operation portion 6. The treatment instrument insertion opening 13 is formed so as to allow a treatment instrument to be inserted into a subject through a treatment instrument insertion tube passage (not shown in the drawing) formed in the insertion portion 2 and the operation portion 6.

The operation portion 6 includes: a vertical bending operation knob 7 which bends a bending portion 4 (described later) of the insertion portion 2 in a vertical direction; and a lateral bending operation knob 9 which bends the bending portion 4 in a lateral direction.

The operation portion 6 further includes: a fixing lever 8 which fixes a rotary position of the vertical bending operation knob 7; and a fixing knob 10 which fixes a rotary position of the lateral bending operation knob 9.

The insertion portion 2 includes a distal end portion 3, the bending portion 4, and a flexible tube portion 5 which are arranged in this order from a distal end side, and is formed in an elongated shape.

The bending portion 4 is bent in a plurality of directions, for example, in four directions, that is, in the upward, downward, leftward, and rightward directions due to rotary operations of the vertical bending operation knob 7 and the lateral bending operation knob 9 mounted on the operation portion 6. With such operations, the bending portion 4 can change an observation direction of an observation optical system (not shown in the drawing) which is mounted in the distal end portion 3, and can enhance inserting ability of the distal end portion 3 into a subject.

The flexible tube portion 5 is connected to a proximal end side of the bending portion 4 in the longitudinal axis direction N (hereinafter, simply referred to as a proximal end side), and has flexibility to an extent that the flexible tube portion 5 can be bent in a passive manner.

As shown in FIG. 2 to 5, an exterior 6g of the operation portion 6 is formed of: a first exterior member 30 which is positioned on a bend preventing member 15 side and on which the treatment instrument insertion opening 13 is formed; and a second exterior member 50 which is disposed at a proximal end of the first exterior member 30, that is, at a position disposed adjacently to the first exterior member 30 in the longitudinal axis direction N. Various knobs 7, 9, and 10 and the lever 8 described above are mounted on the second exterior member 50.

As shown in FIG. 3 to FIG. 5, the first exterior member 30 and the second exterior member 50 are respectively formed in an elongated barrel shape in the longitudinal axis direction N.

As shown in FIG. 3 to FIG. 5 and FIG. 7, a small diameter part 51 having a diameter narrowed in an inside direction KI with respect to a radial direction K is formed on a distal end side of the second exterior member 50, and the second exterior member 50 is connected to the first exterior member 30 due to engagement of the small diameter part 51 with an inner periphery of the first exterior member 30 on a proximal end side.

An O-shaped ring groove 51w is formed in a circumferential shape on an outer peripheral surface 51g of the small diameter part 51 in a circumferential direction C. An O-shaped ring 60 which is a water tightness maintaining member is fitted in the O-shaped ring groove 51w.

Due to the provision of the O-shaped ring 60 fitted in the O-shaped ring groove 51w, when the small diameter part 51 is fitted and coupled in an inner periphery of the first exterior member 30 on a proximal end side, watertight sealing is provided between an inner peripheral surface 30n of the first exterior member 30 and the outer peripheral surface 51g of the small diameter part 51.

The O-shaped ring 60 is disposed more inside than a sealing member 40 described later in the radial direction K.

A radius of the second exterior member 50 changes at a part of the outer peripheral surface 51g of the small diameter part 51 disposed at a position on a more proximal end side than the O-shaped ring groove 51w. A sealing member fitting groove 51v is formed circumferentially on this part.

The sealing member 40 which is elastically deformable and has a ring-shape is fitted in the sealing member fitting groove 51v in an exposed manner to the outside, the sealing member fitting groove 51v being disposed at a connecting portion F which is a connection part between the first exterior member 30 and the second exterior member 50 in the longitudinal axis direction N.

In other words, the sealing member 40 fitted in the sealing member fitting groove 51v is a member which, when the second exterior member 50 is connected to the first exterior member 30, seals a gap formed between a proximal end surface 30k of the first exterior member 30 and an end surface 50s of the sealing member fitting groove 51v of the second exterior member 50 in the radial direction K in the longitudinal axis direction N. Such sealing is provided by the elastic deformation of the sealing member 40 generated by compression of the sealing member 40 by the proximal end surface 30k and the end surface 50s in the longitudinal axis direction N.

In this case, as shown in FIG. 8, a compressive cross-sectional area B of the sealing member 40 may preferably be formed smaller than a space cross-sectional area A defined by the sealing member 40, an outer peripheral surface 30g of the first exterior member 30 and an outer peripheral surface 50g of the second exterior member 50. With such a configuration, when the sealing member 40 is compressed in the longitudinal axis direction N, there is no possibility that the sealing member 40 projects from the sealing member fitting groove 51v in an outside direction KO with respect to the radial direction K relative to the outer peripheral surfaces 30g, 50g and hence, an external appearance of the connecting portion F becomes smooth whereby the external appearance of the connecting portion F is enhanced.

As shown in FIG. 9, an inner peripheral surface 40n of the sealing member 40 disposed circumferentially is formed into a shape where, in a non-compressed state, only two protruding portions 40d which protrude in the inside direction KI with respect to the radial direction K and face each other are brought into contact with a bottom surface of the sealing member fitting groove 51v.

In other words, when the sealing member 40 disposed circumferentially is in a non-compressed state, the inner peripheral surface 40n is disposed with a gap S formed between the sealing member 40 and the bottom surface of the sealing member fitting groove 51v except for two protruding portions 40d.

The reason is as follows. When the sealing member 40 is compressed in the longitudinal axis direction N, a diameter enlarging direction of the sealing member 40 with respect to the radial direction K is guided in the inside direction KI due to the gap S, that is, the gap S absorbs the compression of the sealing member 40. Accordingly, it is possible to prevent the occurrence of a phenomenon that a diameter of the sealing member 40 is enlarged in the outside direction KO so that the sealing member 40 projects from the sealing member fitting groove 51v in the outside direction KO whereby an external appearance of the connecting portion F can be enhanced.

Further, only two protruding portions 40d which face each other are brought into contact with the inner peripheral surface 40n and hence, positioning of the sealing member 40 in the sealing member fitting groove 51v in a circumferential direction C can be easily performed.

To enhance an external appearance of the connecting portion F, the sealing member 40 may be integrally formed with either the first exterior member 30 or the second exterior member 50.

In this case, a seam between the sealing member 40 and the first exterior member 30 or the second exterior member 50 becomes smooth when the sealing member 40 is integrally formed with the first exterior member 30 or the second exterior member 50 and hence, the external appearance of the connecting portion F is enhanced.

The sealing member 40 is formed with an elastic modulus smaller than an elastic modulus of the first exterior member 30 and an elastic modulus of the second exterior member 50.

The reason is as follows. In a case where the elastic modulus of the sealing member 40 is set higher than the elastic modulus of the first exterior member 30 and the elastic modulus of the second exterior member 50, when the sealing member 40 is compressed in the longitudinal axis direction N by the proximal end surface 30k and the end surface 50s, the first exterior member 30 and the second exterior member 50 are deformed.

In other words, with the above-mentioned configuration, the deformation of the first exterior member 30 and the second exterior member 50 can be prevented and hence, the external appearance of the connecting portion F can be enhanced.

As shown in FIG. 6 and FIG. 10, a protruding portion 52 which protrudes in the inside direction KI with respect to the radial direction K and has a circumferential shape in a circumferential direction C is formed on a distal end of an inner peripheral surface 51n of the small diameter part 51 in the longitudinal axis direction N (hereinafter, simply referred to as the distal end).

A recessed portion 53 is formed in a part of the protruding portion 52 in the circumferential direction C. The recessed portion 53 has a shape where a side of the recessed portion 53 in the inside direction KI is closed, and the recessed portion 53 penetrates the protruding portion 52 in the longitudinal axis direction N.

A plurality of recessed portions 53 may be formed in the protruding portion 52 in the circumferential direction C.

Further, as shown in FIG. 5, FIG. 7, and FIG. 12, after the first exterior member 30 is connected to the second exterior member 50, with respect to the inner peripheral surface 30n of the first exterior member 30 on a proximal end side, at a position in the circumferential direction C which faces the recessed portion 53 in the longitudinal axis direction N which is also the position disposed in a spaced-apart manner from the sealing member 40 in the radial direction K and in the longitudinal direction N, more specifically, at the position on a more distal end side than the sealing member 40 and the O-shaped ring 60, two convex portions 31, 32 and two longitudinal positioning convex portions 33 are respectively formed. These convex portions 31, 32 and longitudinal positioning convex portions 33 are formed as positioning members for positioning the first exterior member 30 with respect to the second exterior member 50 in the longitudinal axis direction N and the circumferential direction C along with abutting and contacting of the first exterior member 30 to the second exterior member 50.

The convex portions 31, 32, and two longitudinal positioning convex portions 33 are formed in an elongated shape in the longitudinal axis direction N, and are formed in a shape projecting from the inner peripheral surface 30n in the inside direction KI with respect to the radial direction K.

When the first exterior member 30 and the second exterior member 50 are connected to each other, as shown in FIG. 7 and FIG. 12, the respective end surfaces 33m of two longitudinal positioning convex portions 33 abut against a distal end surface 52m of the protruding portion 52 so that the first exterior member 30 is positioned with respect to the second exterior member 50 in the longitudinal axis direction N.

With such a configuration, it is possible to prevent the occurrence of a phenomenon that the sealing member 40 is excessively compressed so that a compression amount is increased whereby the sealing member 40 projects from the outer peripheral surfaces 30g, 50g in the outside direction KO.

As shown in FIG. 11, notched portions 31h, 32h are respectively formed on the convex portions 31, 32 such that the diameters of the convex portions 31, 32 become small on a distal end side in the outside direction KO with respect to the radial direction K. As a result, protruding projections 31i, 32i are respectively formed on the convex portions 31, 32.

The protruding projections 31i, 32i engage with the recessed portion 53 by fitting when the first exterior member 30 and the second exterior member 50 are connected to each other.

As shown in FIG. 6 and FIG. 12, notched surfaces 31m, 32m which are formed by the notched portions 31h, 32h of the convex portions 31, 32 form abutting surfaces which abut against the distal end surface 52m of the protruding portion 52 when the protruding projections 31i, 32i engage with the recessed portion 53.

Due to abutting of the notched surfaces 31m, 32m against the distal end surface 52m, when the first exterior member 30 and the second exterior member 50 are connected to each other, the first exterior member 30 is positioned with respect to the second exterior member 50 in the longitudinal axis direction N.

With such a configuration, it is possible to prevent the occurrence of a phenomenon that the sealing member 40 is excessively compressed so that a compression amount is increased whereby the sealing member 40 projects from the outer peripheral surfaces 30g, 50g in the outside direction KO.

Positioning of the first exterior member 30 with respect to the second exterior member 50 may be performed using only the above-mentioned longitudinal positioning convex portions 33 without using the convex portions 31, 32. Conversely, positioning of the first exterior member 30 with respect to the second exterior member 50 may be performed using only the convex portions 31, 32 without using the longitudinal positioning convex portions 33.

As shown in FIG. 6, FIG. 10, and FIG. 12, outer peripheral side surfaces 31d, 32d of the protruding projections 31i, 32i in the outside direction KO with respect to the radial direction K form contact surfaces which are brought into contact with respective opening end surfaces 52r of the recessed portion 53 substantially parallel to the longitudinal axis direction N when the protruding projections 31i, 32i engage with the recessed portion 53.

Since the outer peripheral side surfaces 31d, 32d are brought into contact with the respective opening end surfaces 52r, when the first exterior member 30 and the second exterior member 50 are connected to each other, the first exterior member 30 is positioned with respect to the second exterior member 50 in the circumferential direction C.

The reason why the positioning member is formed of two convex portions such as the convex portions 31, 32 is as follows. With the use of two convex portions, a strength of the positioning member can be increased with certainty compared to the case where one convex portion is used. Accordingly, the positioning member may be formed of one convex portion in the case where such requirement can be ignored.

In the case where a plurality of recessed portions 53 are formed with respect to the protruding portions 52 in a circumferential direction C, two convex portions 31, 32 may be formed on the inner peripheral surface 30n for each recessed portion 53.

Other configurations are equal to the corresponding configurations of the conventional exterior 6g.

In this manner, according to this embodiment, the case is described where the first exterior member 30 and the second exterior member 50 are connected to each other and, thereafter, positioning of the first exterior member 30 with respect to the second exterior member 50 in the longitudinal axis direction N and the circumferential direction C is performed by making the convex portions 31, 32 engage with the recessed portion 53 at a position other than the connecting portion F, more specifically, at the position in a spaced-apart manner in the inside direction KI. with respect to the radial direction K relative to the sealing member 40 and also in a spaced-apart manner toward a distal end side in the longitudinal axis direction N relative to the sealing member 40 and the O-shaped ring 60.

In this manner, the configuration where positioning is performed in the vicinity of the sealing member 40 and the O-shaped ring 60 at the connecting portion F is not adopted and hence, it is possible to prevent a diameter of the connecting portion F from becoming large-sized due to the positioning member.

Positioning of the first exterior member 30 with respect to the second exterior member 50 in the longitudinal axis direction N can be performed with certainty by making the respective notched surfaces 31m, 32m abut against the distal end surface 52m and hence, a compression amount of the sealing member 40 can be controlled with high accuracy. Further, positioning of the first exterior member 30 with respect to the second exterior member 50 in the circumferential direction C can be performed with certainty by bringing the outer peripheral side surfaces 31d, 32d into contact with the respective opening end surfaces 52r.

In this manner, since there is no possibility that the first exterior member 30 is displaced with respect to the second exterior member 50 in the longitudinal axis direction N and in the circumferential direction C, that is, since the positioning accuracy can be enhanced, excessive compression of the sealing member 40 can be prevented. Accordingly, an external appearance of the endoscope 1 at the connecting portion F can be enhanced.

As has been described above, it is possible to provide the endoscope 1 having the configuration where the first exterior member 30 can be positioned with respect to the second exterior member 50 in the longitudinal axis direction N and in the circumferential direction C with a favorable external appearance without increasing the diameter of the connecting portion F.

In the above-mentioned embodiment, the exterior 6g has been described by taking the case where the exterior 6g is firmed of two exterior members consisting of the first exterior member 30 and the second exterior member 50 as an example. Needless to say, the present invention is applicable to a connection structure where three or more exterior members are connected to each other.

In the above-mentioned embodiment, the configuration is described where the small diameter part 51 of the second exterior member 50 is inserted into the first exterior member 30. However, as an opposite case, the present invention is also applicable to a configuration where a small diameter part formed on the proximal end side of the first exterior member 30 is inserted into the second exterior member 50.

The insertion apparatus is described by exemplifying the endoscope. However, the present invention is not limited to such an endoscope. Needless to say, the present invention is applicable to other insertion apparatuses such as a treatment instrument.

The exterior of the insertion apparatus is also not limited to the exterior 6g of the operation portion 6 of the endoscope 1. Needless to say, the present invention is applicable to the connection structure of other exterior members of the endoscope 1, and the connection structure of exterior members of the insertion apparatus other than the endoscope.

What is claimed is:

1. An insertion apparatus comprising:
a first exterior member;
a second exterior member disposed at a position where the second exterior member is adjacent to the first exterior member in a longitudinal axis direction of the first exterior member;
a sealing member disposed at a connection part between the first exterior member and the second exterior member in the longitudinal axis direction, the sealing member being configured to deform elastically; and
a positioning member disposed in a spaced-apart manner from the sealing member in the longitudinal axis direction or in a circumferential direction around a longitudinal axis of the first exterior member, the positioning member being configured to position the first exterior member with respect to the second exterior member in the longitudinal axis direction and in the circumferential direction.

2. The insertion apparatus according to claim 1, wherein the positioning member is mounted on the first exterior member, and is configured to position the first exterior member with respect to the second exterior member in the longitudinal axis direction and in the circumferential direction by making the positioning member abut against the second exterior member and bringing the positioning member into contact with the second exterior member.

3. The insertion apparatus according to claim 1, wherein the first exterior member and the second exterior member are formed in a barrel shape, and
the positioning member has an abutting surface which abuts against the second exterior member in the longitudinal axis direction, and a contact surface which is brought into contact with the second exterior member in the circumferential direction.

4. The insertion apparatus according to claim 3, wherein the positioning member is formed of a convex portion which is formed on an inner peripheral surface of the first exterior member in a protruding manner, the convex portion forming the abutting surface and the contact surface, and
the convex portion is configured to position the first exterior member with respect to the second exterior member due to engagement of the convex portion with a recessed portion formed on the second exterior member.

5. The insertion apparatus according to claim 1, wherein an elastic modulus of the sealing member is smaller than an elastic modulus of the first exterior member and an elastic modulus of the second exterior member.

6. The insertion apparatus according to claim 2, wherein the second exterior member includes a small diameter part, a diameter of which is made small in a radial direction, at the connection part, and
the small diameter part is fitted inside the first exterior member.

7. The insertion apparatus according to claim 6, wherein the positioning member is mounted on the first exterior member, the positioning member being configured to abut against and to be brought into contact with the small diameter part.

8. The insertion apparatus according to claim 1, wherein the sealing member is disposed in a sealing member fitting groove formed on the connection part.

9. The insertion apparatus according to claim 8, wherein a gap is formed between the sealing member and the sealing member fitting groove in a radial direction in a state where the sealing member is not compressed.

10. The insertion apparatus according to claim 8, wherein the sealing member is provided such that a cross-sectional area of the sealing member along a longitudinal axis is set smaller than a cross-sectional area of the sealing member fitting groove along the longitudinal axis in a state where the sealing member is compressed.

11. The insertion apparatus according to claim 1, further comprising: an insertion portion extending along the longitudinal axis and being inserted into a subject; and an operation portion connected to a proximal end of the insertion portion along the longitudinal axis, wherein
the first exterior member and the second exterior member form an exterior of the operation portion.

12. The insertion apparatus according to claim 11, further comprising a water tightness maintaining member disposed more inside the sealing member in a radial direction for providing watertight sealing between the first exterior member and the second exterior member in the radial direction, wherein
the positioning member is disposed in a spaced-apart manner from the sealing member and the water tightness maintaining member in a direction toward a distal end of the insertion portion with respect to the longitudinal axis direction.

13. A connection structure of an exterior member comprising:
a first exterior member;
a second exterior member disposed at a position where the second exterior member is adjacent to the first exterior member in a longitudinal axis direction of the first exterior member;
a sealing member disposed at a connection part between the first exterior member and the second exterior member in the longitudinal axis direction, the sealing member being configured to deform elastically; and
a positioning member disposed in a spaced-apart manner from the sealing member in the longitudinal axis direction or in a circumferential direction around a longitudinal axis of the first exterior member, the positioning member configured to position the first exterior member with respect to the second exterior member in the longitudinal axis direction and in the circumferential direction.

14. An endoscope comprising:
an operation portion which includes a first exterior member, a second exterior member disposed at a position where the second exterior member is adjacent to the first exterior member in a longitudinal axis direction of the first exterior member, and a sealing member disposed at a connection part between the first exterior member and the second exterior member in the longitudinal axis direction, the sealing member being configured to deform elastically; and
an insertion portion connected to the operation portion on a first exterior member side, wherein
the first exterior member and the second exterior member include a positioning member disposed in a spaced-apart manner from the sealing member in the longitudinal axis direction or in a circumferential direction around a longitudinal axis of the first exterior member, the positioning member configured to position the first exterior member with respect to the second exterior member in the longitudinal axis direction and in the circumferential direction.

15. The endoscope according to claim 14, wherein the first exterior member includes a treatment instrument insertion opening into which a treatment instrument is inserted, and
the second exterior member includes a bending operation knob configured to perform a bending operation of a bending portion of the insertion portion.
